(19) United States
(12) Patent Application Publication
(10) Pub. No.: US 2008/0103398 A1
(43) Pub. Date: May 1, 2008

(54) Pulse Taking and Spectral Analysis and Display Instrument

(76) Inventor: Chin Ming Huang, Taichung City (TW)

Correspondence Address: Dr. BANGER SHIA, 102 Lindencrest Ct., Sugar Land, TX, 77479-5201, US

(21) Appl. No.: 11/555,198

(22) Filed: Oct. 31, 2006

(51) Int. Cl. A61B 5/02

(52) U.S. Cl. 600/500

(57) ABSTRACT

A pulse taking and spectral analysis and display instrument comprises a movable device, a sensing device, a signal processing device, and a computer. The position of the sensing device in the Z-axis, Y-axis and X-axis directions can be adjusted by the moveable device, thus improving the degree of sensitivity and the convenience of operation. The sensing device is used to detect the pulse signal at the patient's radial artery, and the pulse signal is to be transmitted to and processed by the signal processing device. The computer serves to calculate the Fourier transform and the Power spectrum of the digital signal, making a thorough analysis of the change in the spectral of the respective pulse signals. And finally, the wave pulse diagram, spectral diagram and the power spectral data are displayed on the display.

### BACKGROUND OF THE INVENTION

1. Field of the Invention

The present invention relates to an analysis display, and more particularly to a pulse taking and spectral analysis and display instrument.

2. Description of the Prior Art

A doctor of traditional Chinese medicine usually takes a patient's pulse by placing three fingers (index finger, middle finger, and ring finger) of an arbitrary hand on the radial artery of the patients' wrist with varying pressures, so as to carry out an analysis of the pulse by feeling the reaction of the blood vessel with his fingers analyzing the pulse. When taking a pulse, the pressure applied and the tactile sensitivity cannot be clearly defined since they vary with individual doctors. Therefore, only a doctor of traditional Chinese medicine with numerous experiences in various diseases can make a correct analysis of pulse, and an inexperienced doctor can't do it correctly. For the current medical pulse diagnosis instruments, for example, when a pulse diagnosis instrument is making a pulse diagnosis to check the condition of a patient's stomach, although the pulse wave signal can be shown on the display (as shown in FIGS. 1 and 2), the pulse signal cannot be analyzed. Accordingly, the pulse diagnosis instrument is unable to provide an objective analysis on the vital energy and the state of the blood. As a result, the analysis capabilities cannot be improved effectively, and the creditability of the pulse analysis will be impaired.

The present invention has arisen to mitigate and/or obviate the afore-described disadvantages.

### SUMMARY OF THE INVENTION

The primary objective of the present invention is to provide a pulse taking and spectral analysis and display instrument. The sensing device of the present invention is used to detect the pulse signal at the patient's radial artery, and the pulse signal is to be transmitted to and processed by the signal processing device. The computer serves to calculate the Fourier transform and the Power spectrum of the digital signal, making a thorough analysis of the change in the spectral of the respective pulse signals. The computer serves to display the wave pulse diagram, spectral diagram and the power spectral data, thus providing an explicit pulse analysis and display quickly, and effectively improving the reliability of the pulse analysis.

The second objective of the present invention is to provide a pulse taking and spectral analysis and display instrument, wherein the position of the sensing device in the Z-axis, Y-axis and X-axis directions can be adjusted by the moveable device, thus not only improving the degree of sensitivity and the convenience of operation, but also making the sensing device in exact contact with the patient's radial artery so as to sense the pulse signal thereof. Therefore, it facilitates the comparison analysis of the pulse signal.

### BRIEF DESCRIPTION OF THE DRAWINGS

FIG. 1 is a pulse wave graph in accordance with the present invention of showing the normal state of the stomach;

FIG. 2 is a pulse wave graph in accordance with the present invention of showing an abnormal state of the stomach;

FIG. 3 is an assembly view of showing that a moveable device, a sensing device, a signal processing device of a pulse taking and spectral analysis and display instrument in accordance with an embodiment of the present invention are used with a desktop computer;

FIG. 4 is an assembly view in accordance with the present invention of showing the moveable device and the sensing device;

FIG. 5 is an amplified view in accordance with the present invention of showing a part of the pulse sensor, the Y-axis adjusting assembly, and the X-axis adjusting assembly;

FIG. 6 is an amplified view in accordance with the present invention of showing a part of the Y-axis adjusting assembly, and the X-axis adjusting assembly;

FIG. 7 is an enlarged illustrative view in accordance with the present invention of showing sensing device;

FIG. 8 is an assembly view of showing that a moveable device, a sensing device, a signal processing device of a pulse taking and spectral analysis and display instrument in accordance with the present invention are used with a notebook computer;

FIG. 9 is a flow chart in accordance with the present invention;

FIG. 10 is an operative view in accordance with the present invention;

FIG. 11 is a spectral display within 0-50 Hz in accordance with the present invention of showing the result of the pulse analysis after the pulse taking and spectral analysis and display instrument takes the pulse of a patient whose stomach is healthy;

FIG. 12 is a spectral display within 0-50 Hz in accordance with the present invention of showing the result of the pulse analysis after the pulse taking and spectral analysis and display instrument takes the pulse of a patient whose stomach is unhealthy;

FIG. 13 is a spectral amplification display within 13-50 Hz in accordance with the present invention of showing the result of the pulse analysis after the pulse taking and spectral analysis and display instrument takes the pulse of a patient whose stomach is healthy; and

FIG. 14 is a spectral amplification display within 13-50 Hz in accordance with the present invention of showing the result of the pulse analysis after the pulse taking and spectral analysis and display instrument takes the pulse of a patient whose stomach is unhealthy.

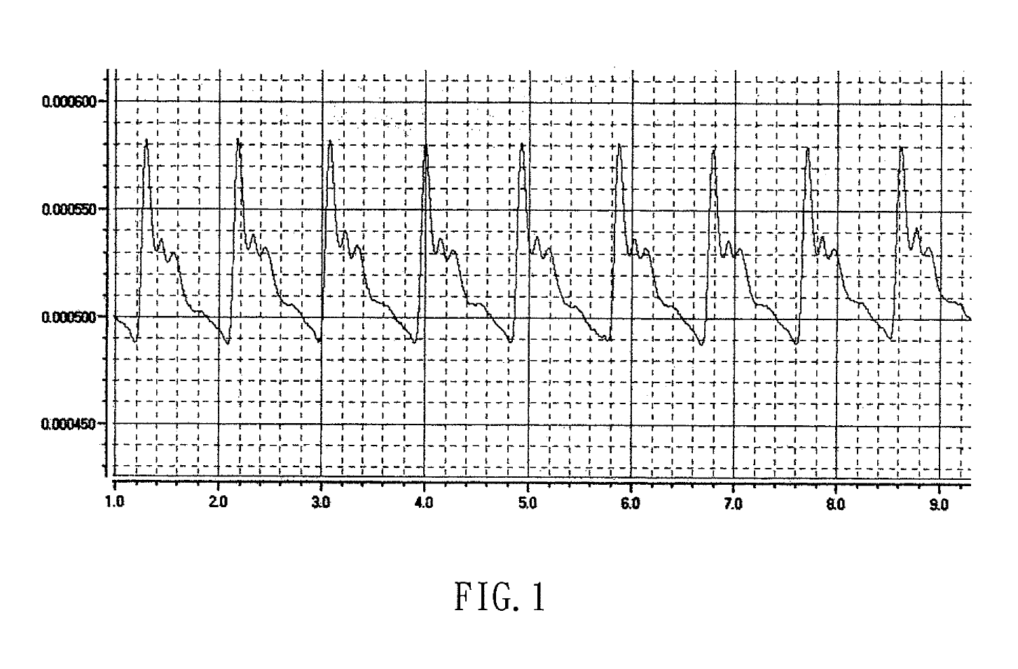

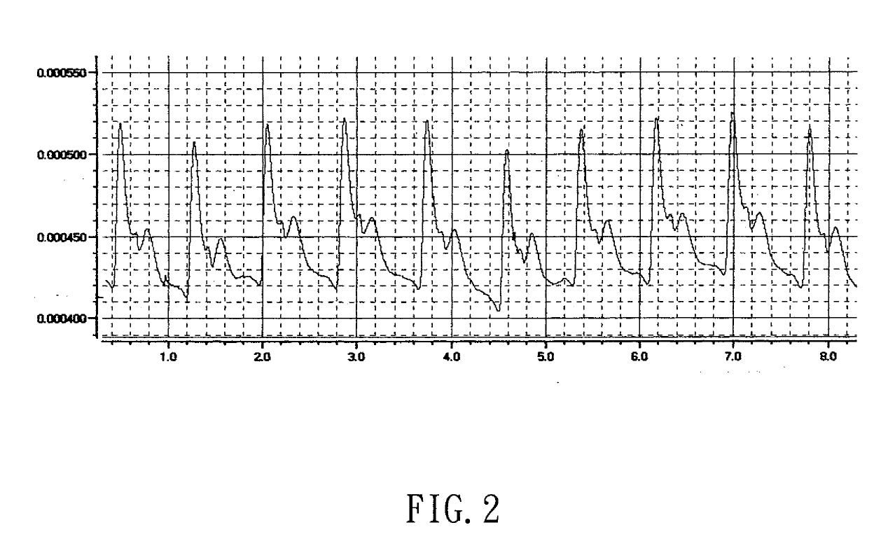

### DETAILED DESCRIPTION OF THE PREFERRED EMBODIMENTS

The present invention will be more clear from the following description when viewed together with the accompanying drawings, which show, for purpose of illustrations only, the preferred embodiment in accordance with the present invention.

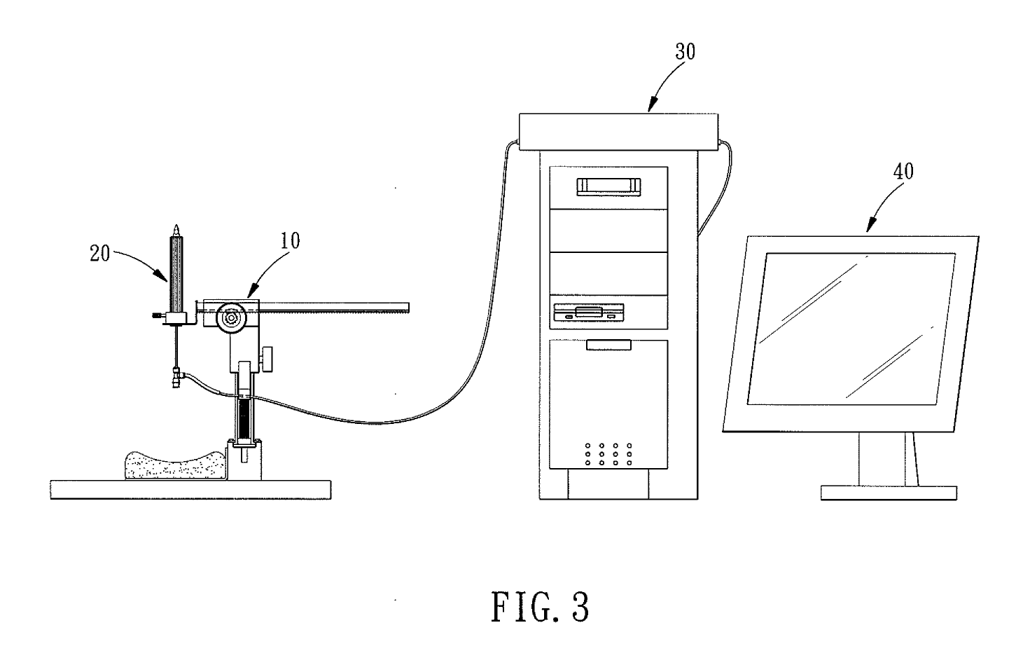

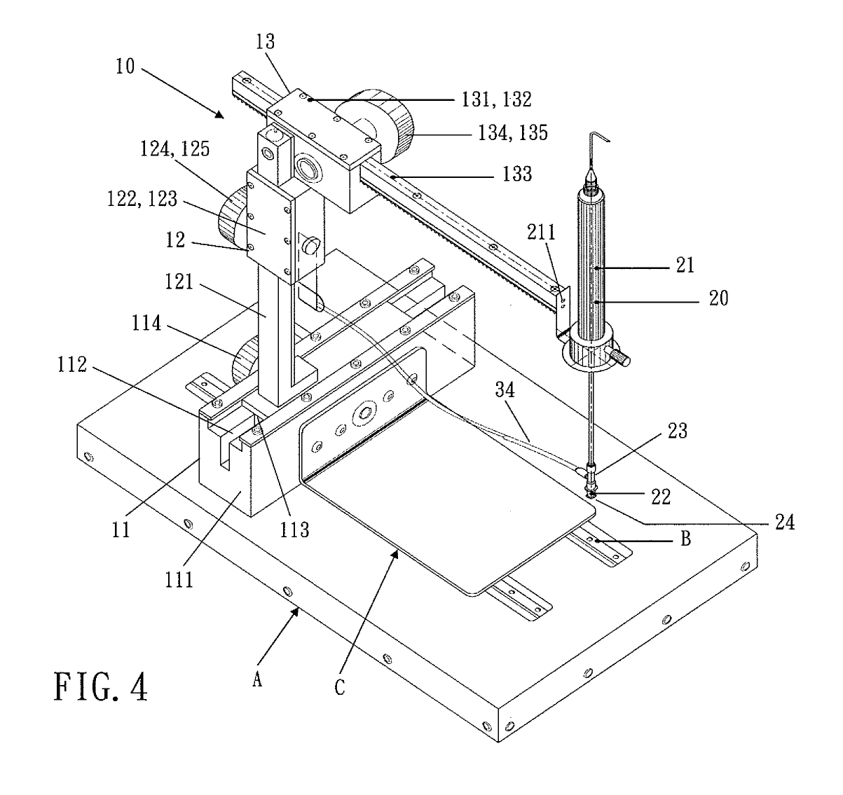

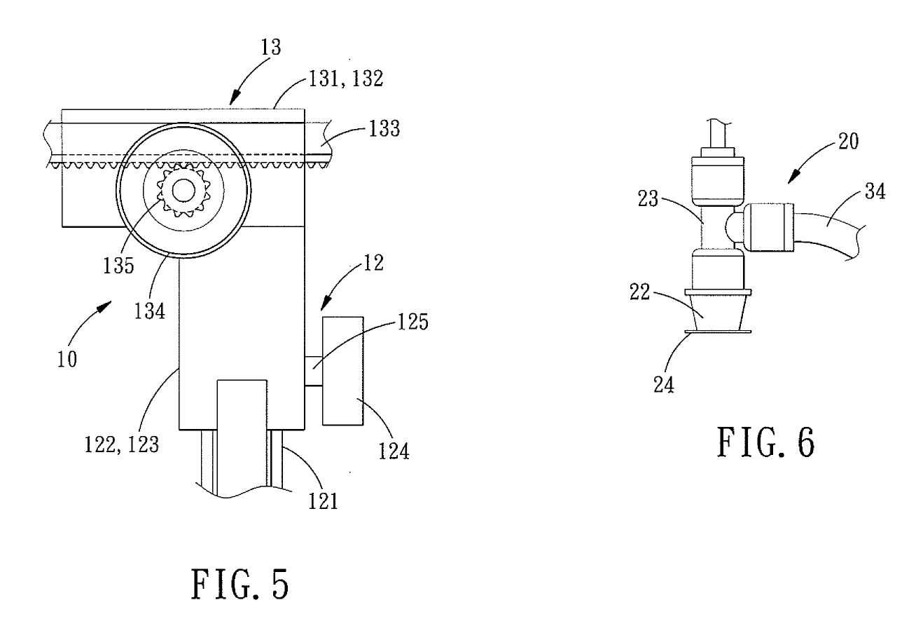

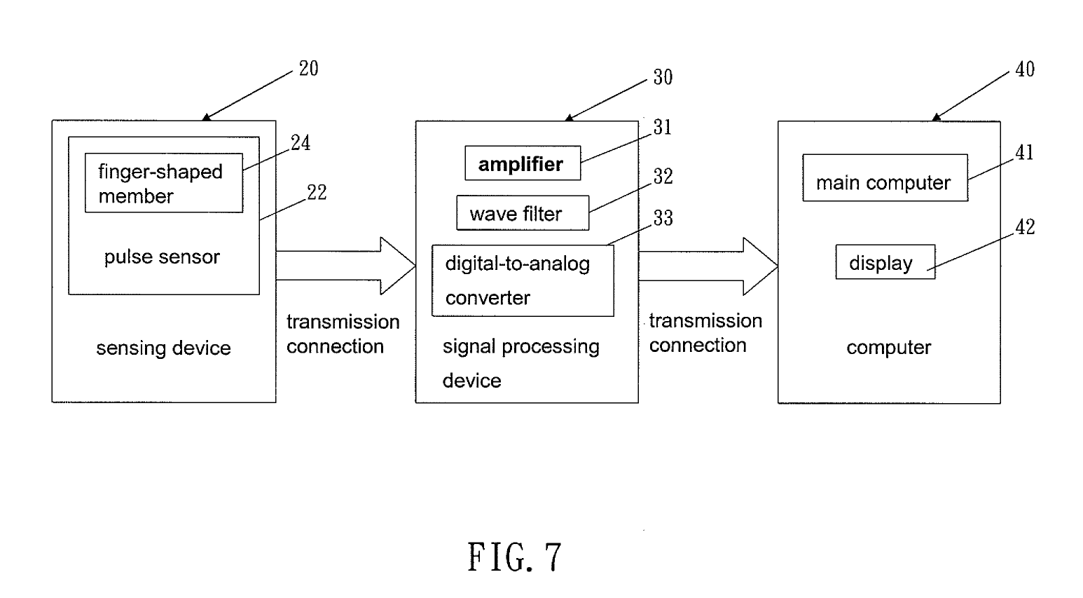

Referring to FIG. 3, which is an assembly view of showing that a moveable device, a sensing device, a signal processing device of a pulse taking and spectral analysis and display instrument in accordance with an embodiment of the present invention (also with reference to FIGS. 4, 5, 6 and 7) are used with a desktop computer. The pulse taking and spectral analysis and display instrument essentially comprises a base A, a pair of slide rails B, a carrying board C, a movable device 10, a sensing device 20, a signal processing device 30, and a computer 40.

The pair of slide rails B, the carrying board C, and the movable device 10 are disposed on the top surface A1 of the base A.

The slide rails B (are linear rails) are parallel to each other and are symmetrically engaged in the top surface of the base A.

The carrying board C is fixed on the top surface of the slide rails B.

The movable device 10 includes a Z-axis adjusting assembly 11, a Y-axis adjusting assembly 12 and an X-axis adjusting assembly 13.

The Z-axis adjusting assembly 11 includes a Z-axis slide seat 111 having a Z-axis groove 112, a Z-axis toothed rack 113, and a Z-axis adjusting disc 114 having a Z-axis toothed shaft (not shown).

The Z-axis slide seat 111 is horizontally fixed at one side of the carrying board C, and the slide rails B enable the carrying board C and the Z-axis slide seat 111 to slide horizontally.

The Z-axis groove 112 is T-shaped in cross section and is formed in the top surface of the Z-axis slide seat 111.

The Z-axis toothed rack 113 is T-shaped and is received in the Z-axis groove 112.

The Z-axis adjusting disc 114 is axially formed with a Z-axis toothed shaft that is pivotally connected at one side of the Z-axis slide seat 111 and is received in the Z-axis groove 112. The toothed shaft of the Z-axis adjusting disc 114 is meshed with the Z-axis toothed rack 113. When the Z-axis adjusting disc 114 rotates, the Z-axis toothed shaft will synchronously drive the Z-axis toothed rack 113 to move along the Z-axis groove 112.

The Y-axis adjusting assembly 12 includes a Y-axis toothed rack 121, a Y-axis positioning seat 122 having a guiding hole 123, and a Y-axis adjusting disc 124 with a Y-axis toothed shaft 125.

The Y-axis toothed rack 121 has one end vertically fixed to an end of the top surface of the Z-axis toothed rack 113.

The Y-axis positioning seat 122 is fixed to the Y-axis toothed rack 121 via the guiding hole 123.

The Y-axis adjusting disc 124 is axially formed with a Y-axis toothed shaft 125 that is to be pivotally connected to one side of the Y-axis positioning seat 122 and received in the guiding hole 123. The Y-axis toothed shaft 125 is meshed with the Y-axis toothed rack 121. When the Y-axis adjusting disc 124 rotates, the Y-axis toothed shaft 125 will drive the Y-axis toothed rack 121 to move. The Y-axis positioning seat 122 is movable along the Y-axis toothed rack 121 via the guiding hole 123.

The X-axis adjusting assembly 13 includes an X-axis positioning seat 131 having a guiding hole 132, an X-axis toothed rack 133, and an X-axis adjusting disc 134 having an X-axis toothed shaft 135.

One side of the X-axis positioning seat 131 is vertically fixed to the top surface of the Y-axis positioning seat 122 and is located adjacent to the Y-axis toothed rack 121.

The X-axis toothed rack 133 is inserted in the guiding hole 132 of the X-axis positioning seat 131.

The X-axis adjusting disc 134 is axially formed with an X-axis toothed shaft 135 that is to be pivotally connected to one side of the X-axis positioning seat 131 and received in the guiding hole 132. The X-axis toothed shaft 135 is meshed with the X-axis toothed rack 133. When the X-axis adjusting disc 134 rotates, the X-axis toothed shaft 135 will drive the X-axis toothed rack 133 to move along the guiding hole 132 of the X-axis positioning seat 131.

The sensing device 20 includes a force gauge 21 and a pulse sensor 22.

A L-shaped strengthening piece 211 is arranged at the mid of the force gauge 21 and has one end fixed to one end of the X-axis toothed rack 133 of the X-axis adjusting assembly 13. The force gauge 21 is vertical to the X-axis toothed rack 133 of the X-axis adjusting assembly 13. The force gauge 21 serves to provide a pushing force less than 500 grams to the base A and is arranged in parallel to the Y-axis toothed rack 121 of the Y-axis adjusting assembly 12.

The pulse sensor 22 includes a T-shaped connector 23 with one end fixed to the bottom of the force gauge 21. At the end of the connector 23 opposite the force gauge 21 is fixed a laminar finger-shaped member 24, and the finger-shaped member 24 faces the base A and is an imitation of human being's index finger, middle finger and ring finger.

The signal processing device 30 includes an amplifier 31, a wave filter 32, and a digital-to-analog converter 33.

The signal processing device 30 is further provided with a transmission cable 34 having an end connected to the end of the connector 23 neighboring the force gauge 21 and serves to receive the pulse signal transmitted from the transmission cable 20. The pulse signal is amplified by the amplifier 31 and is denoised by the wave filter 32, and then the digital-to-analog converter 33 serves to convert analog signal into digital signal.

The computer 40 (desktop type) includes a main computer 41 and a display 42.

The main computer module 41 is connected to the signal processing device 30, and in the main computer module 41 is installed a conversion software (C++ Builder 6.0) that serves to calculate the Fourier transform and the Power spectrum after receiving the pulse signal transmitted from the pulse sensor 22.

The display 42 is provided for displaying the wave pulse diagram, spectral diagram and the power spectral data.

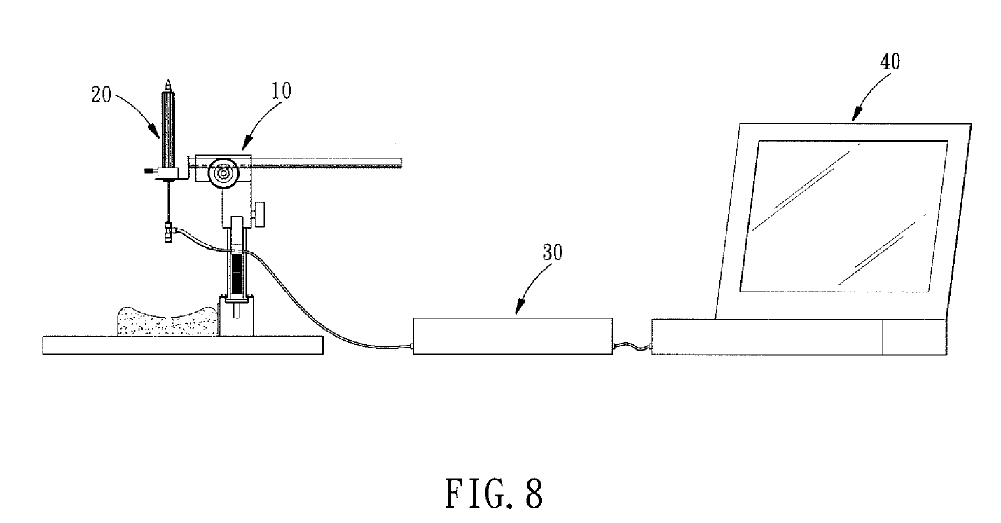

FIG. 8 is an assembly view of showing that a moveable device, a sensing device, a signal processing device of a pulse taking and spectral analysis and display instrument in accordance with the present invention are used with a notebook computer 40.

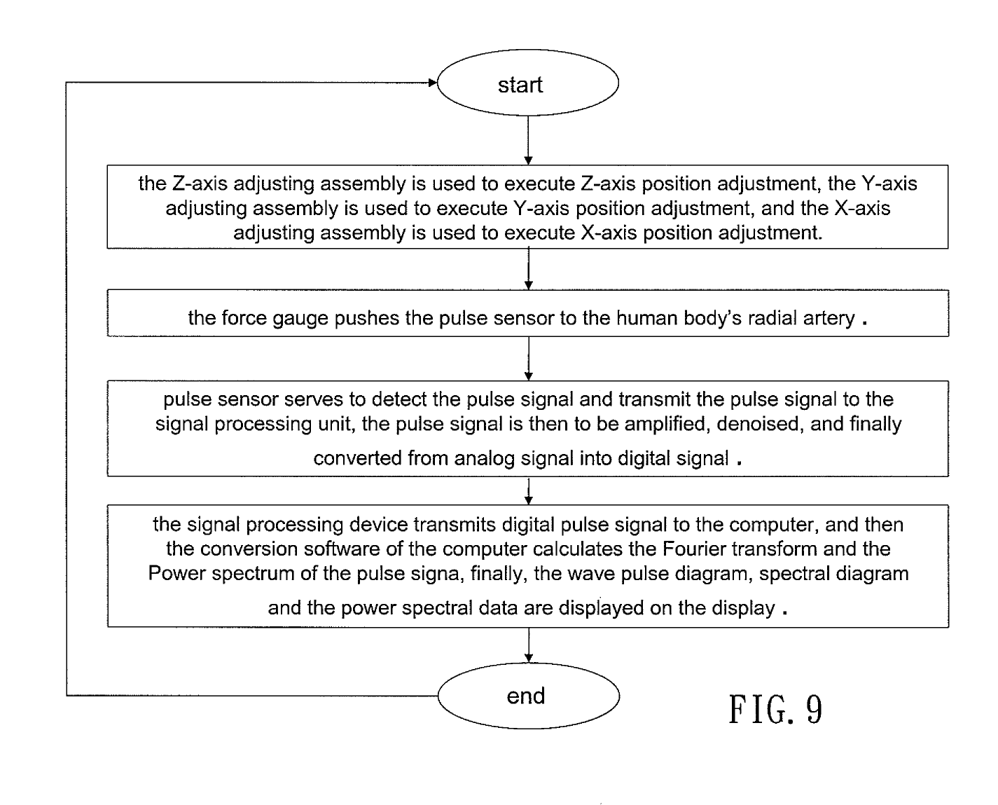

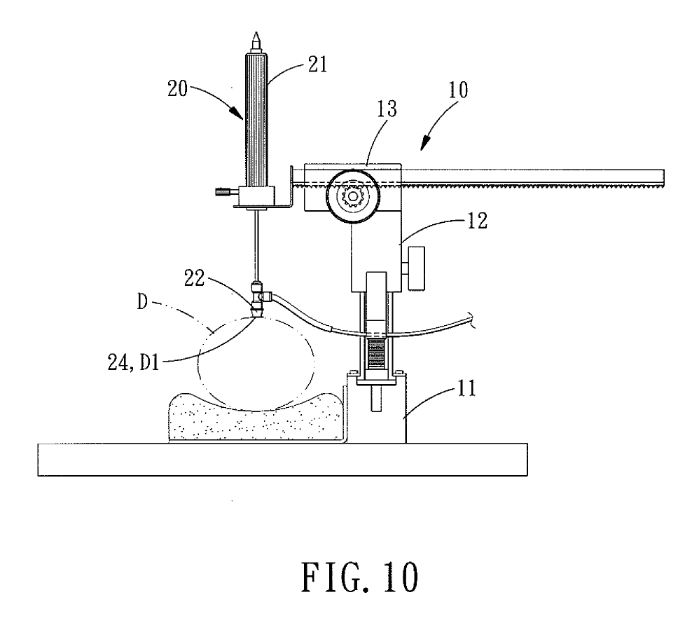

For a better understanding of the present invention, its operation and function, reference should be made to FIGS. 9 and 10.

To use the pulse taking and spectral analysis and display instrument, the signal processing device 30 and the computer 40 should be switched on initially, and thus the conversion software of the main computer module 41 is activated, and the display 42 serves to display signal. After that, the horizontal position of the carrying board C is firstly adjusted by moving it along the slide rails B on the base A, and then the Z-axis adjusting assembly 11 executes Z-axis position adjustment, the Y-axis adjusting assembly 12 executes Y-axis position adjustment, and the X-axis adjusting assembly 13 executes X-axis position adjustment.

When the Z-axis adjusting disc 114 of the Z-axis adjusting assembly 11 rotates, the Z-axis toothed shaft will drive the Z-axis toothed rack 113 to move along within the Z-axis groove 112.

When the Y-axis adjusting disc 124 of the Y-axis adjusting assembly 12 rotates, the Y-axis toothed shaft 125 will be moved with respect to the Y-axis toothed rack 121, so that the Y-axis positioning seat 122 will move along the Y-axis toothed rack 121 via the guiding hole 123.

When the X-axis adjusting disc 134 of the X-axis adjusting assembly 13 rotates, the X-axis toothed shaft 135 will drive the X-axis toothed rack 133 to move in the X-axis direction along the guiding hole 132 of the X-axis positioning seat 131.

After the Z-axis position, the Y-axis position, and the X-axis position are adjusted, the force gauge 21 will push the pulse sensor 22 of the sensing device 20, as shown in FIG. 10, making the finger-shaped member 24 touch the radial artery D1 of the patients' wrist D and detect the pulse signal. And the pulse signal will be transmitted to the signal processing unit 30 by the transmission cable 34. After that the pulse signal will be amplified by the amplifier 31, denoised by the wave filter 32, and converted from analog signal into digital signal by the digital-to-analog converter 33, and finally will be transmitted to the computer 40. The conversion software in the main computer module 41 of the computer 40 will calculate the Fourier transform and the Power spectrum of the pulse signal after receiving the pulse signal transmitted from the pulse sensor 22, making a thorough analysis of the change in the spectral of the respective pulse signals. And finally, the wave pulse diagram, spectral diagram and the power spectral data will be displayed on the display 42.

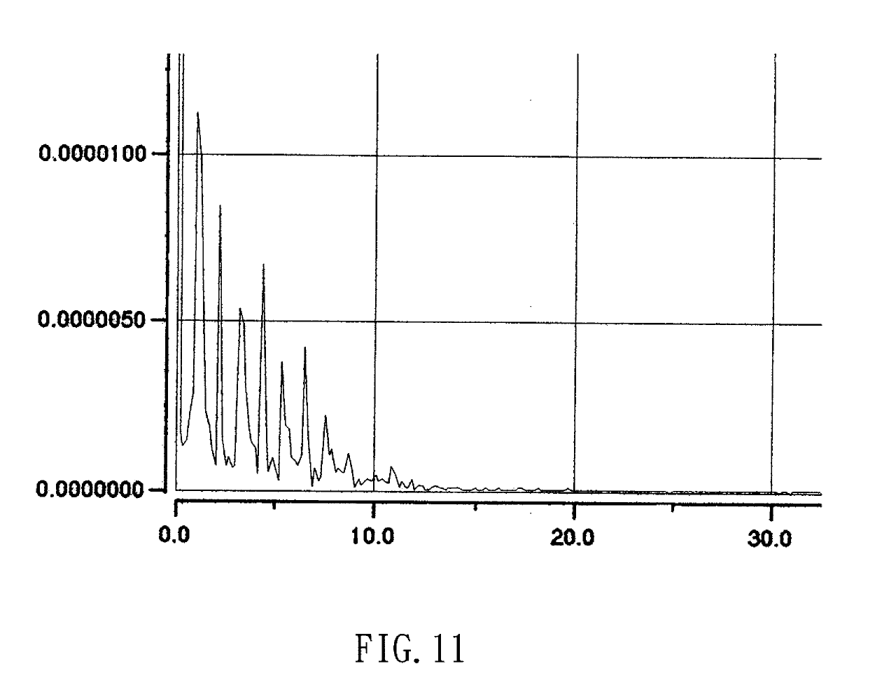

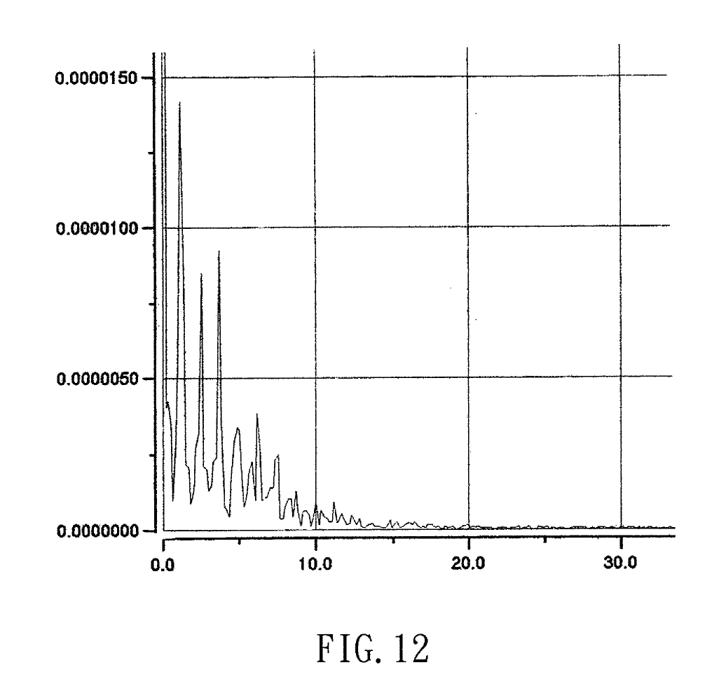

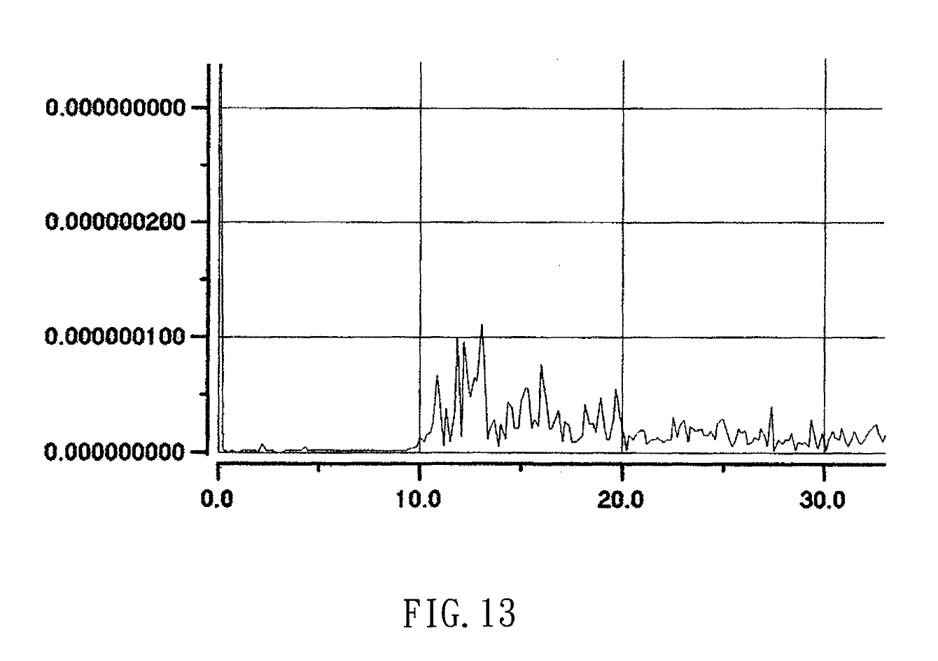

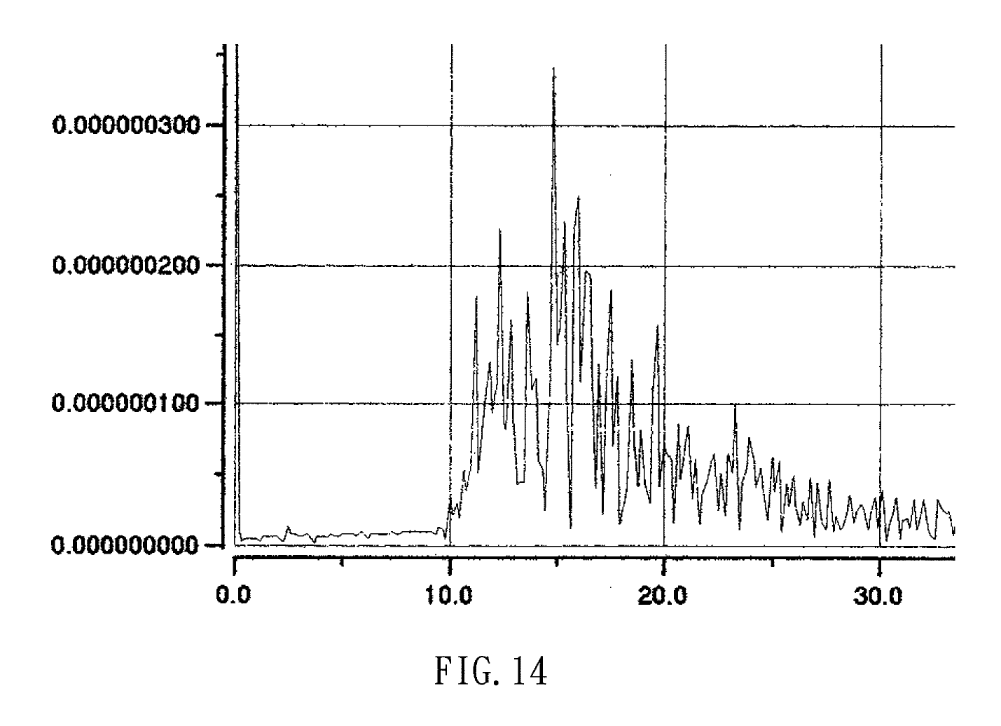

What follows are the sampling and analysis of the normal state and the abnormal state of the stomach, as shown in FIGS. 11 and 12, a 0-50 Hz spectral display carried out by the conversion software of the computer 40, and as shown in FIGS. 12 and 13, a spectral amplification within 13-50 Hz made by the conversion software of the computer 40. The difference between the normal state and the abnormal state of the stomach can be easily detected through comparison of the spectral displays, thus providing an explicit analysis quickly.

To summarize, the pulse taking and spectral analysis and display instrument of the present invention essentially comprises a movable device, a sensing device, a signal processing device, and a computer. The position of the sensing device in the Z-axis, Y-axis and X-axis directions can be adjusted by the moveable device, thus improving the degree of sensitivity and the convenience of operation. The sensing device is used to detect the pulse signal at the patient's radial artery, and the pulse signal is to be transmitted to and processed by the signal processing device. The computer serves to calculate the Fourier transform and the Power spectrum of the digital signal, making a thorough analysis of the change in the spectral of the respective pulse signals. And finally, the wave pulse diagram, spectral diagram and the power spectral data are displayed on the display.

While we have shown and described various embodiments in accordance with the present invention, it is clear to those skilled in the art that further embodiments may be made without departing from the scope of the present invention.

What is claimed is:

1. A pulse taking and spectral analysis and display instrument comprising:
a moveable device disposed on a top surface of a base and employed to perform axial adjusting movement;
a sensing device including a force gauge and a pulse sensor;
the force gauge has one end fixed to the moveable device and has another end located opposite the top surface of the base;
the pulse sensor has one end fixed to the another end of the force gauge, the force gauge enables the pulse sensor to produce a pushing force toward the top surface of the base, another end of the pulse sensor is to be placed on a patient's radial artery to sense the pulse signal; and
a computer connected to the pulse sensor of the sensing device, a conversion software being installed in the computer and serving to carry out spectral analysis display of the pulse signal sensed by the pulse sensor.

2. The pulse taking and spectral analysis and display instrument as claimed in claim 1, wherein the movable device includes a Z-axis adjusting assembly, a Y-axis adjusting assembly and an X-axis adjusting assembly.

3. The pulse taking and spectral analysis and display instrument as claimed in claim 2, wherein a slide rail is engaged in the top surface of the base, and on a top surface of the slide rail is fixed a carrying board.

4. The pulse taking and spectral analysis and display instrument as claimed in claim 3, wherein the Z-axis adjusting assembly is horizontally fixed at one side of the carrying board and serves to perform Z-axial adjusting movement.

5. The pulse taking and spectral analysis and display instrument as claimed in claim 4, wherein the Z-axis adjusting assembly includes a Z-axis slide seat having a Z-axis groove, a Z-axis toothed rack, and a Z-axis adjusting disc having a Z-axis toothed shaft;
the Z-axis slide seat is horizontally fixed at one side of the carrying board;
the Z-axis groove is formed in a top surface of the Z-axis slide seat;
the Z-axis toothed rack is received in the Z-axis groove; and
the Z-axis adjusting disc is axially formed with the Z-axis toothed shaft that is pivotally connected at one side of the Z-axis slide seat and is received in the Z-axis groove, the toothed shaft of the Z-axis adjusting disc is meshed with the Z-axis toothed rack.

6. The pulse taking and spectral analysis and display instrument as claimed in claim 5, wherein the Z-axis toothed rack is formed in a shape similar to the shape of the Z-axis groove.

7. The pulse taking and spectral analysis and display instrument as claimed in claim 4, wherein the Y-axis toothed rack has one end vertically fixed to an end of a top surface of the Z-axis toothed rack and serves to execute adjusting movement in Y-axis direction.

8. The pulse taking and spectral analysis and display instrument as claimed in claim 7, wherein the Y-axis adjusting assembly includes a Y-axis toothed rack, a Y-axis positioning seat having a guiding hole, and a Y-axis adjusting disc with a Y-axis toothed shaft;
the Y-axis toothed rack has one end vertically fixed to an end of the top surface of the Z-axis toothed rack;
the Y-axis positioning seat is fixed to the Y-axis toothed rack via the guiding hole; and
the Y-axis adjusting disc is axially formed with the Y-axis toothed shaft that is to be pivotally connected to one side of the Y-axis positioning seat and received in the guiding hole, the Y-axis toothed shaft is meshed with the Y-axis toothed rack.

9. The pulse taking and spectral analysis and display instrument as claimed in claim 7, wherein the X-axis adjusting assembly has one end vertically fixed to another end of the Y-axis adjusting assembly and serves to perform adjusting movement in X-axis direction.

10. The pulse taking and spectral analysis and display instrument as claimed in claim 9, wherein the X-axis adjusting assembly includes an X-axis positioning seat having a guiding hole, an X-axis toothed rack, and an X-axis adjusting disc having an X-axis toothed shaft;
one side of the X-axis positioning seat is vertically fixed to a top surface of the Y-axis positioning seat and is located adjacent to the Y-axis toothed rack;
the X-axis toothed rack is inserted in the guiding hole of the X-axis positioning seat; and
the X-axis adjusting disc is axially formed with the X-axis toothed shaft that is to be pivotally connected to one side of the X-axis positioning seat and received in the guiding hole, the X-axis toothed shaft is meshed with the X-axis toothed rack.

11. The pulse taking and spectral analysis and display instrument as claimed in claim 3, wherein the slide rail is a linear slide rail.

12. The pulse taking and spectral analysis and display instrument as claimed in claim 1, wherein the sensing device further includes a finger-shaped member fixed on another end of the pulse sensor.

13. The pulse taking and spectral analysis and display instrument as claimed in claim 1, wherein the computer is a desktop computer or a notebook computer.

14. The pulse taking and spectral analysis and display instrument as claimed in claim 1, wherein the conversion software is C++ Builder 6.0.

15. The pulse taking and spectral analysis and display instrument as claimed in claim 1 further comprising a signal processing device which includes an amplifier, a wave filter, and a digital-to-analog converter, the signal processing device is connected to the pulse sensor of the sensing device and the computer and serves to process signal.
